(19) United States
(12) Patent Application Publication
(10) Pub. No.: US 2004/0120935 A1
(43) Pub. Date: Jun. 24, 2004

(54) Genetically modified yt cell line and use thereof

(76) Inventor: Gabriele Pecher, Berlin (DE)

Correspondence Address: BIRCH STEWART KOLASCH & BIRCH, PO BOX 747, FALLS CHURCH, VA, 22040-0747, US

(21) Appl. No.: 10/476,007

(22) PCT Filed: Apr. 18, 2002
(86) PCT No.: PCT/DE02/01437

(30) Foreign Application Priority Data
Apr. 28, 2001 (DE) 101 21 113.9

(51) Int. Cl. A61K 48/00; C12N 5/08

(52) U.S. Cl. 424/93.21; 435/372

(57) ABSTRACT

The invention relates to a YT cell line, modified by genetic engineering, into which the genes of receptors are introduced and which is suitable for treating tumours, tumour metastases, virus infections, for cleaning (purging) blood stem cell preparations and also for diagnostic purposes, Areas of application include the medical field and the pharmaceutical industry. The aim of the invention is to identify tumour cells and other target cells by means of the gene transfer of specific receptors into the YT cell line. An additional aim is to cause a lysis of the identified tumour cells and other target cells, using the newly created cell line. The inventive cell line contains one or more receptors (consisting of variable regions of tumour-specific or target cell specific antibodies (single chain Pv fragments)) and optionally a signal chain, which is derived from the zεta-chain of the human T-cell receptor.

### DESCRIPTION

The invention relates to a genetically modified YT cell line, into which receptor genes have been integrated and can be used, for example, in the treatment and diagnostics of tumours, tumour metastases, autoimmune diseases, viral infections, and for cleaning (purging) of preparations of blood stem cells. Fields of application are in medicine and the pharmaceutical industry.

The genuine YT cell line, which has not been genetically modified, was first described by Yodoi et al. (Yodoi-J; Teshigawara-K; Nikaido-T; Fukui-K; Noma-T; Honjo-T; Takigawa-M; Sasaki-M; Minato-N; Tsudo-M, et-al. TCGF (IL 2)-receptor inducing factor(s). I. Regulation of IL 2 receptor on a natural killer-like cell line (YT cells). J-Immunol. March 1985; 134(3): 1623-30). The cell line was established from the blood of a patient. It is per se not capable of specifically recognising and lysing tumours or other target cells.

The objective of the invention is to achieve the recognition of tumour cells and other target cell types by means of transferring genes encoding specific receptors into the YT cell line. A further objective consists in using the established cell line to accomplish the lysis of the recognised target cells.

The invention will be realised in accordance with the patent claims. The cell line will be genetically modified and can subsequently be used in diagnostics and therapeutics. According to the invention, the genes encoding the receptors which specifically recognise tumour cells and other target cells will be transferred into the cell line by means of genetic engineering. These receptor constructs contain a domain composed of the recognising part of an antibody, which specifically recognises the tumour cells and other target cells, or partial thereof. A signal chain, derived from the zeta-chain of the human T cell receptor, is coupled to this domain and mediates lytic activity.

The advantages of the invention are the following:

The YT cell line is suited for the genetic transfer of receptors and, owing to the transfer of the receptor gene, attains new properties which, for example, consist in the recognition and lysis of tumour cells and other target cells.

The new cell line is immortalised and therefore of unlimited availability.

The cell line can be adapted to various tumour cells and other target cell types by means of selecting the respective receptors accordingly.

The cell line does not lose its efficacy when exposed to gamma-radiation. Radiation impedes further proliferation, without affecting the lethal properties of the cells, so that a sufficient quantity of receptor effects can be anticipated to prevail even after 1-2 days.

The following application examples will serve to farther explain the invention:

### EXAMPLE

A receptor was constructed which specifically recognises the structure of the Carcinoembyonic Antigen (CEA) located on colon cancer cells. The genes of this receptor were integrated into the YT cell line with electroporation as the gene transfer method.

FIG. 1 shows the structural composition of the receptor incorporated into the cell line. One of the essential components of the receptor is the specific single-chain Fv (scFv) fragment. This fragment is made of variable regions of the humanised antibody BW431 which specifically recognises CEA structures. A peptide linker couples the fragment to the zeta-(ζ)-chain of the human T cell receptor that mediates the lytic activity.

The YT cell line which had been equipped with this receptor was then tested for its lytic (killer) activity against tumour cells of the colon. FIG. 2 shows that the YT cell line transfected with the receptor gene lyses the human colon cancer cell lines LS174T and SW1222 as well as the cell line MC32A displaying human CEA on cell surfaces. Control YT cell lines, in which the specific receptor construct mentioned had not been incorporated, but other non-relevant genes (mock, scPhOx-hFcζ) instead, could not significantly lyse the tumour cells in a ⁵¹Cr release assay performed according to common standards.

In addition, the efficiency of tumour lysis resulting from the cell line, which was modified by the transfer of the CEA receptor gene and inhibited in its proliferation by gamma-radiation, was tested in a mouse tumour model. FIG. 3 shows that tumour growth was retarded, when CEA-bearing tumour cells MC32A were applied in the mice together with the genetically modified YT cell line recognising CEA. In comparison, the mice survived longer than the control YT cell line which had been transfected with non-relevant genes and then concomitantly applied with the tumour cells.

The YT cell line thus genetically modified can be used in the therapy of human CEA-bearing tumours, either by applying them directly into the tumour or existing metastases, or by means of systematic intravenous application. The genetically modified YT cell line recognises CEA on the tumours and lyses these tumour cells. Lysis is mediated by the signal chain “built into” the receptor. The cell line can also be used to recognise and lyse tumour cells within preparations of stem cells or other preparations, or, if the cells pertaining to the invention are suitably labelled (e.g., with a dye or radioactive substance), in diagnostics for the detection of tumour cells and metastases, for example, subsequent to intravenous application.

### FIGURE LEGENDS

FIG. 1—Schematic Representation of Specific Fusion Receptors

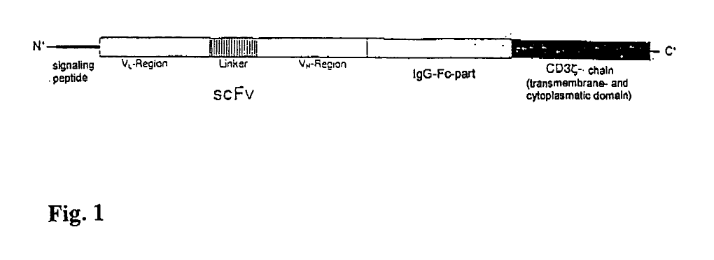

At the N′-terminal of the receptors there is a signal peptide which is required for receptor expression on the surface of YT cells. The receptors consist of a specific single-chain Fv-(scFv-) fragment composed of the variable regions of an antibody (humanised CEA-specific antibodies BW431 in the example shown) joined by a flexible peptide linker, a spacer between the scFv fragment and the cell membrane (the Fc part of the human antibody (IgG-Fc) in the example shown), and the signal-inducing part of the ζ-chain of the human T cell receptor.

FIG. 2—⁵¹Cr Release Assay to Determine the Lysis of CEA-Expressing Tumour Cell Lines by YT Cells, into which the CEA-specific Fusion Receptor scBW431-hFcζ has been Incorporated by means of Gene Transfer.

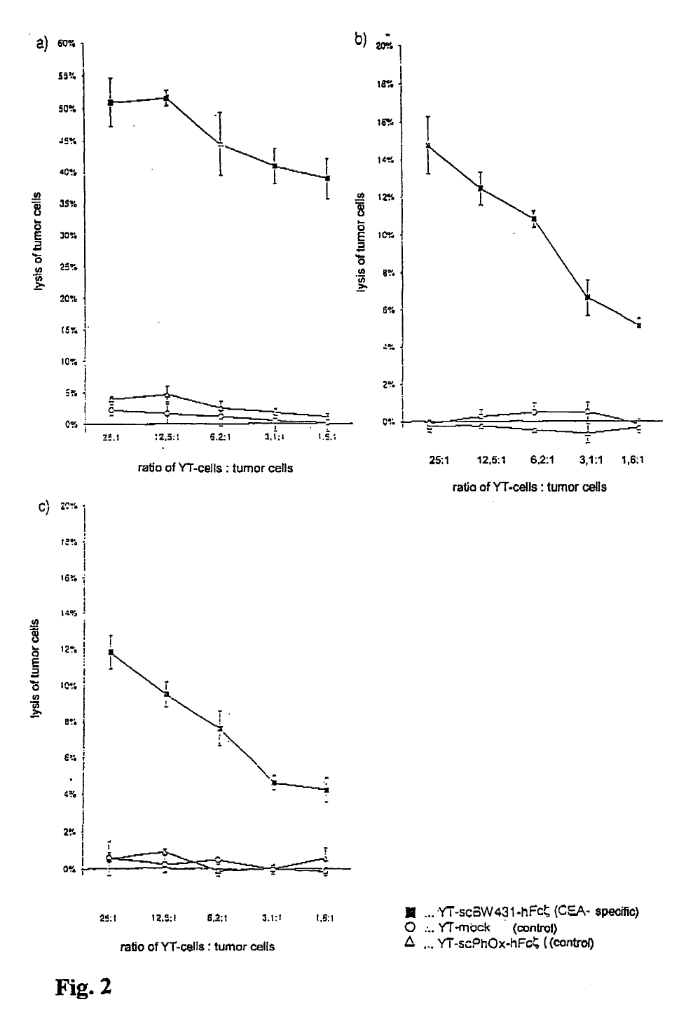

The YT cell line was modified by gene transfer of the receptor scBW431-hFcζ which is specific to human CEA. The cytotoxicity of the YT cells was tested against cell lines a) MC32A, which display human CEA on their surfaces, and against the colon cancer cell lines b) LS174T and c) SW1222. YT cells transfected with a blank vector (mock) or the fusion receptor scPhOx-hFcζ which is unable to recognise tumour cell structures, served as the controls.

FIG. 3—Nod/SCID Mouse Experiment

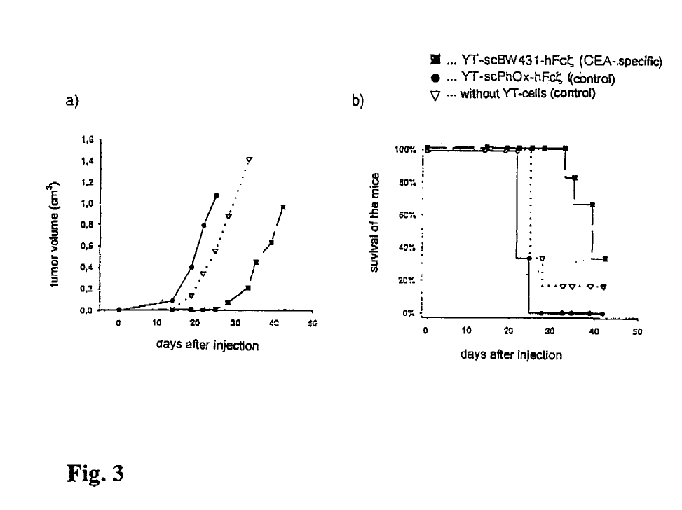

On day 0, MC32A tumour cells were injected together with CEA-specific YT cells (YT-scBW431-hFcζ) or phOx-specific YT cells (YT-scPhOx-hFcζ), or without YT cells under the skin of Nod/SCID mice. The YT cells applied were previously exposed to radiation to prevent further growth. The Figure shows a) tumour growth, and b) the survival rate after the injection of the cells. Animals possessing tumours bigger than 1 cm³ were sacrificed in compliance with animal protection regulations and classified as dead.

1. YT cell line, which is modified by means of gene transfer and can be used in the therapy and diagnostics of human and animal diseases, characterized by the fact that foreign genes are integrated encoding one or several receptors which specifically recognise the respective target cells, and, if necessary, additional genes.

2. YT cell line in accordance with claim 1, characterized by the fact that the receptor contains a recognition domain specifically recognising structures on the respective target cells.

3. YT cell line in accordance with claim 1, characterized by the fact that the receptor contains a recognition domain specifically recognising structures on tumour cells.

4. YT cell line in accordance with claim 1, characterized by the fact that the receptor contains a recognition domain specifically recognising structures on virus-infected cells.

5. YT cell line in accordance with claim 1, characterized by the fact that the receptor contains a recognition domain specifically recognising structures on autoimmune-reactive immune cells.

6. Cell line in accordance with claim 1, characterized by the fact that the receptor domain consists of variable regions of antibodies specific of the tumour or target cells (single chain Fv fragments).

7. Cell line in accordance with claim 1, characterized by the fact that the incorporated receptor additionally contains a signal chain which is capable of mediating a signal to induce cytolytic activity of the cell line YT.

8. Cell line, characterized by the fact that the signal chain of the incorporated receptor is derived from the zeta-chain of the human T cell receptor.

9. Cell line, characterized by the fact that the signal chain of the incorporated receptor is coupled to the recognition domain by means of a linker domain.

10. Cell line, characterized by the fact that the linker domain is derived from the human IgG-Fc part.

11. Cell line in accordance with claim 1, characterized by the fact that the receptor domains bind specifically to the human tumour antigens Carcinoembryonic Antigen (CEA), mucin, or CD33.

12. Cell line in accordance with claim 1, characterized by the fact that the cell line is exposed to gamma radiation to prevent further proliferation.

13. Cell line in accordance with claim 1, characterized by the fact that one or several suicide genes are additionally incorporated into this cell line that makes it susceptible to the action of certain pharmaceuticals/chemotherapeutics/substances.

14. Cell line in accordance with claim 1, characterized by the fact that the genes encoding the receptor construct or receptor constructs, and, if necessary, the suicide gene or suicide genes, are incorporated into the cell line by means of electroporation.

15. Cell line in accordance with claim 1, characterized by the fact that the cell line is labelled with a dye or radioactive substance.
